(19) United States
(12) Patent Application Publication
(10) Pub. No.: US 2025/0042885 A1
(43) Pub. Date: Feb. 6, 2025

(54) SOLID STATE FORMS OF LOTILANER AND PROCESS FOR PREPARATION THEREOF

(71) Applicant: TEVA PHARMACEUTICALS INTERNATIONAL GMBH, Jona (CH)

(72) Inventors: Anantha Rajmohan Muthusamy, Sivakasi (IN); Amit Singh, Greater Noida (IN)

(21) Appl. No.: 18/710,688

(22) PCT Filed: Dec. 9, 2022
(86) PCT No.: PCT/US2022/052335

§ 371 Date: May 16, 2024

(30) Foreign Application Priority Data
Dec. 10, 2021 (IN) 202111057565
Jun. 9, 2022 (IN) 202211033182
Jul. 12, 2022 (IN) 202211039872
Aug. 23, 2022 (IN) 202211048021

(51) Int. Cl. C07D 413/04; A61K 31/422

(52) U.S. Cl. CPC C07D 413/04; A61K 31/422

(57) ABSTRACT

The present disclosure encompasses a solid-state form of Lotilaner, in embodiment processes for preparation thereof, and pharmaceutical compositions thereof. The present disclosure further encompasses Lotilaner salts and their solid state forms, as well as processes for preparation thereof, and pharmaceutical compositions thereof.

### FIELD OF THE DISCLOSURE

The present disclosure encompasses solid state forms of Lotilaner, in embodiments processes for preparation thereof, and pharmaceutical compositions thereof. The present disclosure further encompasses Lotilaner salts and their solid state forms, as well as processes for preparation thereof, and pharmaceutical compositions thereof.

### BACKGROUND OF THE DISCLOSURE

Lotilaner, chemical name 3-Methyl-N-[2-oxo-2-(2,2,2-trifluoroethylamino)ethyl]-5-[(5S)-5-(3,4,5-trichlorophenyl)-5-(trifluoromethyl)-4H-1,2-oxazol-3-yl]thiophene-2-carboxamide, has the following chemical structure:

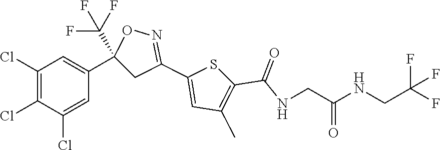 [SMILES: Cc1cc(C2=NO[C@@](c3cc(Cl)c(Cl)c(Cl)c3)(C(F)(F)F)C2)sc1C(=O)NCC(=O)NCC(F)(F)F]

Lotilaner is a veterinary drug administered orally for the treatment and prevention of flea and tick infestations in dogs. Lotilaner has also been investigated for use in the killing of ticks attached to human skin. It is currently developed for the treatment of eye infection and blepharitis.

The compound is described in International Publication No. WO 2010/070068.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Lotilaner.

### SUMMARY OF THE DISCLOSURE

The present disclosure provides solid state forms, particularly crystalline polymorphs, of Lotilaner, processes for preparation thereof, and pharmaceutical compositions thereof. The present disclosure further encompasses Lotilaner salts and their solid state forms, as well as processes for preparation thereof, and pharmaceutical compositions thereof.

These solid state forms, particularly crystalline polymorphs of Lotilaner or Lotilaner salts can be used to prepare other solid state forms of Lotilaner, Lotilaner salts or co-crystals and their solid state forms.

The present disclosure also provides uses of the said solid state form of Lotilaner or Lotilaner salts in the preparation of other solid state forms of Lotilaner, Lotilaner salts or co-crystals salts thereof.

The present disclosure provides solid state forms, particularly crystalline polymorphs of Lotilaner or Lotilaner salt s for use in medicine, including for the treatment of patients with eye infections and/or blepharitis.

The present disclosure also encompasses the use of the solid state forms, particularly crystalline polymorphs, of Lotilaner or Lotilaner salts of the present disclosure for the preparation of pharmaceutical compositions and/or formulations. In embodiments, solid state forms, particularly crystalline polymorphs of Lotilaner or Lotilaner salts of the present disclosure are used to prepare a solution or a suspension for ophthalmic administration.

In another aspect, the present disclosure provides pharmaceutical compositions, such as ophthalmic solution and/or suspension, comprising the solid state forms, particularly crystalline polymorphs of Lotilaner or Lotilaner salts according to the present disclosure.

The present disclosure includes processes for preparing the above mentioned pharmaceutical compositions. The processes include combining any one or a combination of the solid state forms, particularly crystalline polymorphs of Lotilaner or Lotilaner salts with at least one pharmaceutically acceptable excipient.

The solid state forms, particularly crystalline polymorphs of Lotilaner or Lotilaner salts as defined herein and the pharmaceutical compositions or formulations of solid state forms, particularly crystalline polymorphs, of Lotilaner may be used as medicaments, such as for the treatment various types of eye infections and/or blepharitis.

The present disclosure also provides methods of treating various types of eye infections and/or blepharitis, by administering a therapeutically effective amount of any one or a combination of the solid state forms, particularly crystalline polymorphs of Lotilaner or Lotilaner salts of the present disclosure, or at least one of the above pharmaceutical compositions, to a subject suffering from eye infections and/or blepharitis. The present disclosure also provides uses of solid state forms, particularly crystalline polymorphs of Lotilaner or Lotilaner salts of the present disclosure, or at least one of the above pharmaceutical compositions, for the manufacture of medicaments for treating patients with eye infections and/or blepharitis.

### BRIEF DESCRIPTION OF THE DRAWINGS

FIG. 1 shows a characteristic X-ray powder diffraction pattern (XRPD) of amorphous Lotilaner.

FIG. 2 shows a characteristic XRPD of crystalline Lotilaner Form LT1.

FIG. 3 shows a characteristic XRPD of crystalline Lotilaner Form LT2.

FIG. 4 shows a characteristic XRPD of crystalline Lotilaner Form LT3.

FIG. 5 shows a characteristic XRPD of crystalline Lotilaner mono-besylate Form LBSA1.

FIG. 6 shows a characteristic XRPD of crystalline Lotilaner Form LT4.

FIG. 7 shows a characteristic XRPD of crystalline Lotilaner Form LT5.

FIG. 8 shows a characteristic thermogravimetric analysis (TGA) thermogram of crystalline Lotilaner Form LT1.

FIG. 9 shows a characteristic differential scanning calorimetry (DSC) thermogram of crystalline Lotilaner Form LT1.

FIG. 10 shows a characteristic TGA thermogram of crystalline Lotilaner Form LT4.

FIG. 11 shows a characteristic DSC thermogram of crystalline Lotilaner Form LT4.

FIG. 12 shows a characteristic TGA thermogram of crystalline Lotilaner Form LT5.

FIG. 13 shows a characteristic DSC thermogram of crystalline Lotilaner Form LT5.

FIG. 14a shows a characteristic solid-state ¹³C NMR spectrum of crystalline Lotilaner Form LT1, full scan.

FIG. 14b shows a characteristic solid-state ¹³C NMR spectrum of crystalline Lotilaner Form LT1, zoomed-in, in the range of 0-100 ppm.

FIG. 14c shows a characteristic solid-state ¹³C NMR spectrum of crystalline Lotilaner Form LT1, zoomed-in in the range of 100-200 ppm.

FIG. 15a shows a characteristic solid-state ¹³C NMR spectrum of crystalline Lotilaner Form LT4, full scan.

FIG. 15b shows a characteristic solid-state ¹³C NMR spectrum of crystalline Lotilaner Form LT4, zoomed-in in the range of 0-100 ppm

FIG. 15c shows a characteristic solid-state ¹³C NMR spectrum of crystalline Lotilaner Form LT4, zoomed-in in the range of 100-200 ppm

FIG. 16a shows a characteristic solid-state ¹³C NMR spectrum of crystalline Lotilaner Form LT5, full scan.

FIG. 16b shows a characteristic solid-state ¹³C NMR spectrum of crystalline Lotilaner Form LT5, zoomed-in in the range of 0-100 ppm.

FIG. 16c shows a characteristic solid-state ¹³C NMR spectrum of crystalline Lotilaner Form LT5, zoomed-in in the range of 100-200 ppm.

### DETAILED DESCRIPTION OF THE DISCLOSURE

The present disclosure encompasses solid state forms of Lotilaner, particularly crystalline polymorphs of Lotilaner; as well as processes for preparation thereof, and pharmaceutical compositions thereof. The present disclosure further encompasses Lotilaner salts and their solid state forms, as well as processes for preparation thereof, and pharmaceutical compositions thereof.

Solid state properties of Lotilaner or Lotilaner salts and crystalline polymorphs thereof can be influenced by controlling the conditions under which Lotilaner and crystalline polymorphs thereof are obtained in solid form.

The solid state form may be referred to herein as “Lotilaner Form name” or “Crystalline Form name of Lotilaner” or “Crystalline Lotilaner Form name” or “Crystalline polymorph name of Lotilaner” or “Crystalline Lotilaner polymorph name” or “Lotilaner polymorph name”. For example, crystalline Form I of Lotilaner may be interchangeably referred to herein as Lotilaner Form I or as Crystalline Lotilaner Form I or as Crystalline polymorph I of Lotilaner or as Crystalline Lotilaner polymorph I or Lotilaner polymorph I.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. For example, crystalline form @@ of Lotilaner which is polymorphically pure, contains: about 20% (w/w) or less, about 10% (w/w) or less, about 5% (w/w) or less, about 2% (w/w) or less, about 1% (w/w) or less, or about 0% of any other forms of Lotilaner. Thus, a crystalline polymorph of Lotilaner or Lotilaner salt described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Lotilaner or Lotilaner salts. In some embodiments of the disclosure, the described crystalline polymorph of Lotilaner or Lotilaner salt may contain from about 1% to about 20% (w/w), from about 5% to about 20% (w/w), or from about 5% to about 10% (w/w) of one or more other solid state forms of Lotilaner or Lotilaner salt.

A compound may be referred to herein as chemically pure or purified compound or as substantially free of any other compounds. As used herein in this context, the expression “substantially free of any other compounds” will be understood to mean that the pure compound contains about 20% (w/w) or less, about 10% (w/w) or less, about 5% (w/w) or less, about 2% (w/w) or less, about 1% (w/w) or less, or about 0% of any other compound as measured, for example, by HPLC. Thus, pure or purified Lotilaner or Lotilaner salt herein as substantially free of any compounds would be understood to contain greater than about 80% (w/w), greater than about 90% (w/w), greater than about 95% (w/w), greater than about 98% (w/w), greater than about 99% (w/w), or about 100% of the subject Lotilaner. In some embodiments of the disclosure, the described pure or purified Lotilaner may contain from about 1% to about 20% (w/w), from about 5% to about 20% (w/w), or from about 5% to about 10% (w/w) of one or more other compounds.

In specific embodiments, the above described pure or purified Lotilaner may relate to enantiomeric purity, i.e. pure or purified Lotilaner refers to Lotilaner that is substantially free of enantiomers of Lotilaner.

Depending on which other crystalline polymorphs a comparison is made, the crystalline polymorphs of Lotilaner or Lotilaner salts of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density. For example, Forms LT1, LT4 and LT5 are especially stable under high and low relative humidity conditions at different temperatures, stable to grinding and heating, as well as to pressure. Therefore, these forms are particularly suitable for processing into pharmaceutical dosage forms.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Lotilaner referred to herein as being characterized by graphical data “as depicted in” or “as substantially depicted in” a Figure will thus be understood to include any crystal forms of Lotilaner characterized with the graphical data having such small variations, as are well known to the skilled person, in comparison with the Figure.

As used herein, and unless stated otherwise, the term “anhydrous” in relation to crystalline forms of Lotilaner or Lotilaner salt, relates to a crystalline form of Lotilaner which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, the term “isolated” in reference to crystalline polymorph of Lotilaner or Lotilaner salt of the present disclosure corresponds to a crystalline polymorph of Lotilaner or Lotilaner salt that is physically separated from the reaction mixture in which it is formed.

As used herein, unless stated otherwise, the XRPD measurements are taken using copper Kα1 radiation wavelength 1.5418 Å. XRPD peaks reported herein are measured using Cuk α1 radiation, λ=1.5418 Å, typically at a temperature of 25±3° C.

As used herein, unless stated otherwise, ¹³C NMR reported herein are measured at 500 MHz, at a magic angle spinning frequency ωᵣ/2π=11 kHz, preferably at a temperature of at 298 K±3° C.

As used herein, Thermogravimetric analysis (TGA) carried out under nitrogen at a heating rate of 10° C./min up to 350° C.

As used herein, Differential Scanning calorimetry (DSC) was performed under nitrogen, and a heating rate of 10° C./min up to 300° C.

A thing, e.g., a reaction mixture, may be characterized herein as being at, or allowed to come to “room temperature” or “ambient temperature”, often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

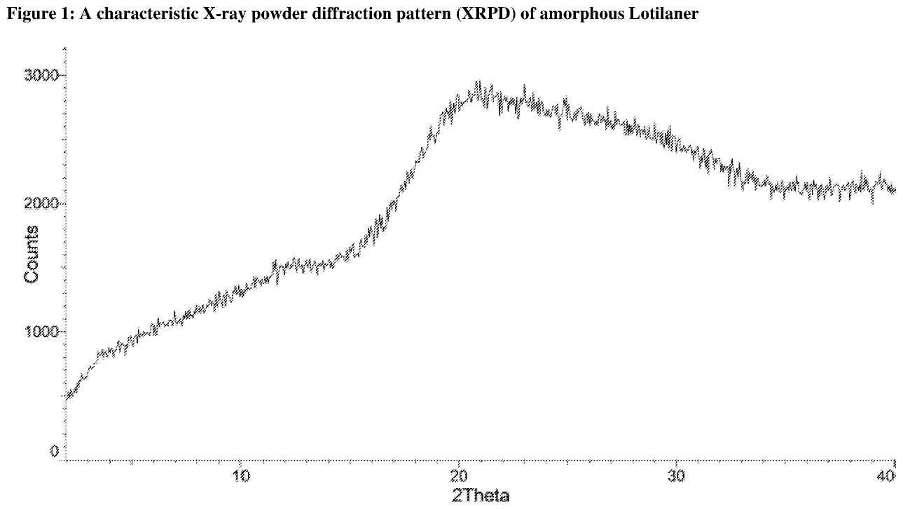

The present disclosure includes amorphous Lotilaner. A typical X-ray powder diffraction pattern of amorphous Lotilaner is presented in FIG. 1.

The present disclosure includes a process for preparing amorphous Lotilaner. The process comprises dissolving Lotilaner in dichloromethane (“DCM”), and distilling the solvent under reduced pressure. Typically, the evaporation is performed at a temperature of about 35° C.-40° C., preferably for a period of from about 30 minutes to about 45 minutes.

The present disclosure includes a crystalline Lotilaner.

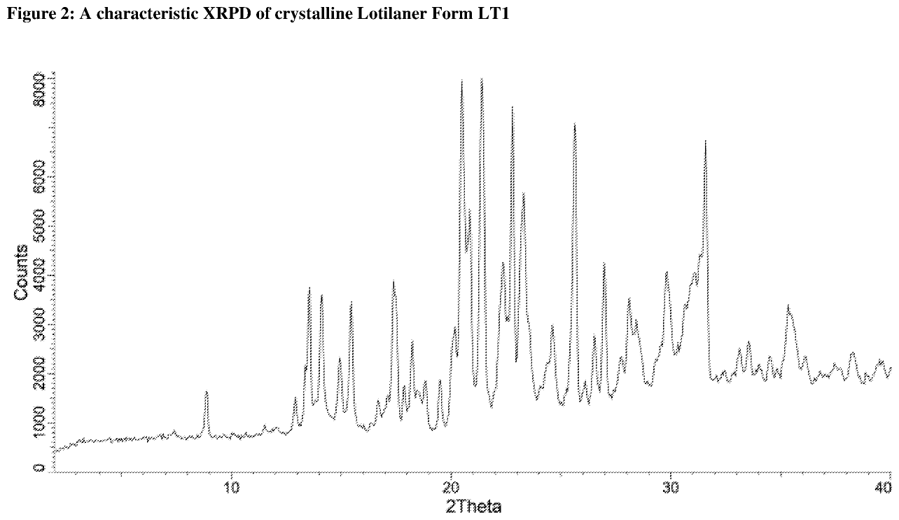

The present disclosure includes a crystalline polymorph of Lotilaner designated Form LT1. The crystalline Form LT1 of Lotilaner may be characterized by data selected from one or more of the following: an X-ray powder diffraction pattern substantially as depicted in FIG. 2; an X-ray powder diffraction pattern having peaks at 13.6, 17.4 and 21.4 degrees 2-theta±0.2 degrees 2-theta; and combinations of these data.

Crystalline Form LT1 of Lotilaner may be further characterized by an X-ray powder diffraction pattern having peaks at 13.6, 17.4 and 21.4 degrees 2-theta±0.2 degrees 2-theta, and also having any one, two, three, four or five additional peaks selected from 14.1, 15.5, 25.6, 27.0 and 31.6 degrees 2-theta±0.2 degrees 2-theta. Alternatively, crystalline Form LT1 of Lotilaner may be characterized by an X-ray powder diffraction pattern having peaks at 13.6, 14.1, 15.5, 17.4, 21.4, 25.6, 27.0 and 31.6 degrees 2-theta±0.2 degrees 2-theta.

Alternatively, Crystalline Form LT1 of Lotilaner may be characterized by an X-ray powder diffraction pattern having peaks at 8.9, 15.0, 21.4, 23.3 and 25.6 degrees 2-theta±0.2 degrees 2-theta. Crystalline Form LT1 of Lotilaner may be further characterized by an X-ray powder diffraction pattern having peaks 8.9, 15.0, 21.4, 23.3 and 25.6 degrees 2-theta±0.2 degrees 2-theta and also having any one, two, three, four or five additional peaks selected from 14.1, 15.5, 25.6, 27.0 and 31.6 degrees 2-theta±0.2 degrees 2-theta. Crystalline Form LT1 of Lotilaner may be further characterized by an X-ray powder diffraction pattern having peaks 8.9, 15.0, 21.4, 23.3 and 25.6 degrees 2-theta±0.2 degrees 2-theta and also having any one, two, three, or four additional peaks selected from 14.1, 15.5, 27.0 and 31.6 degrees 2-theta±0.2 degrees 2-theta.

Crystalline Form LT1 of Lotilaner may be characterized by an X-ray powder diffraction pattern having peaks at 8.9, 13.6, 14.1, 15.0, 15.5, 17.4, 19.5, 21.4, 22.8, 23.3, 24.6, 25.6, 26.5, 27.0 and 31.6 degrees 2-theta±0.2 degrees 2-theta.

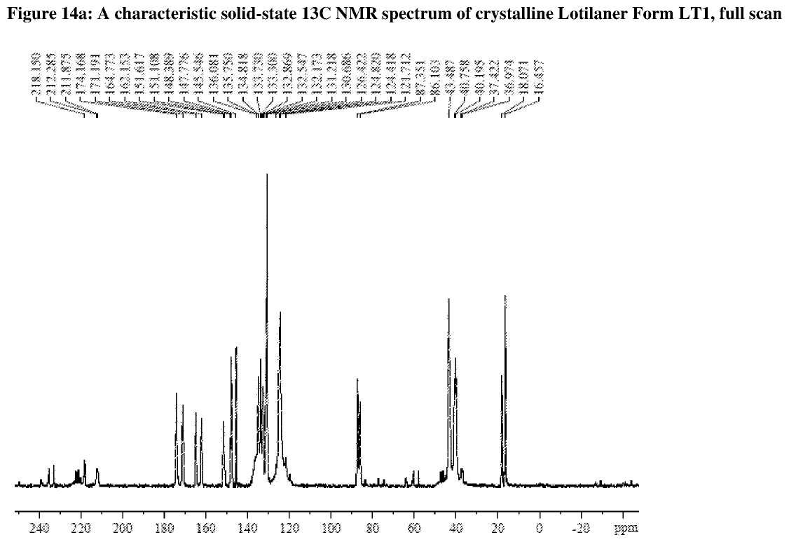

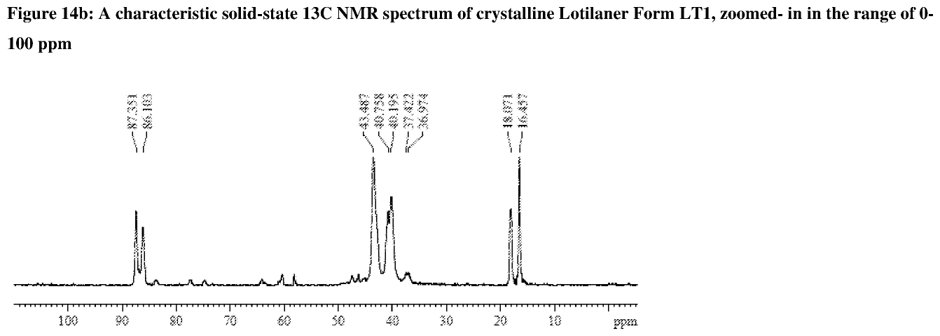

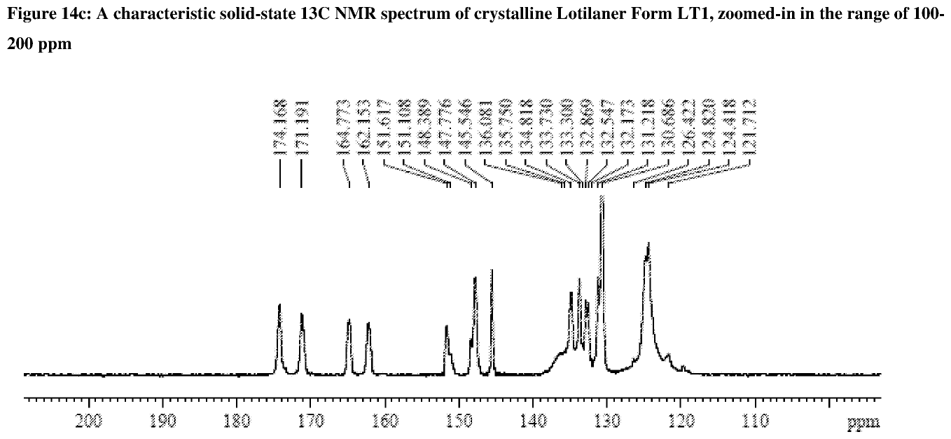

Crystalline Form LT1 of Lotilaner may alternatively or additionally be characterized by a solid state ¹³C NMR spectrum having characteristic peaks at the range of 0-200 ppm at: 18.1, 43.5, 130.7, 147.8, 151.6 and 174.2±0.2 ppm, or a solid-state ¹³C NMR spectrum substantially as depicted in FIG. 14a, 14b or 14c. It may be further characterized by a solid state ¹³C NMR spectrum having characteristic chemical shift absolute differences from a peak at 87.4 ppm±2 ppm of 69.3, 43.9, 43.3, 60.4, 64.2 and 86.8 ppm±0.1 ppm; and or by a solid state ¹³C spectrum having characteristic chemical shift differences from a peak at 174.2 ppm±1 ppm of 86.8 ppm±0.1 ppm

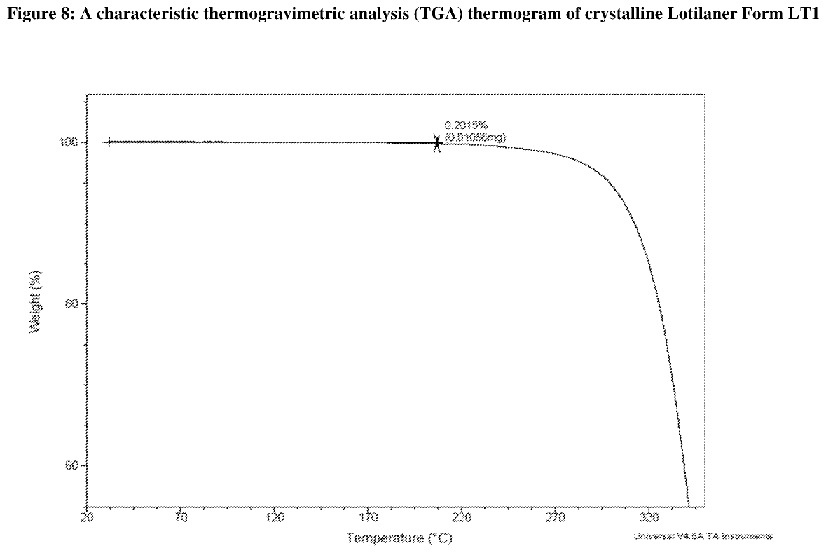

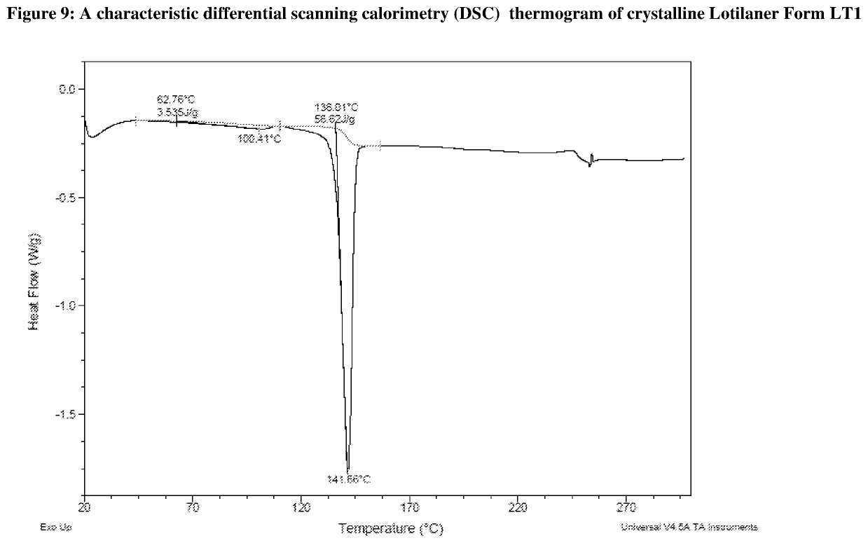

Crystalline Form LT1 of Lotilaner may be characterized by a TGA thermogram substantially as depicted in FIG. 8, a DSC thermogram showing a melting endotherm onset at about 136° C., or by a DSC thermogram substantially as depicted in FIG. 9, or by combination of these data.

In one embodiment of the present disclosure, crystalline Form LT1 of Lotilaner is isolated.

Crystalline Form LT1 of Lotilaner may be an anhydrous form. Typically, the water content in LT1 is less than 1% (w/w), as detected for example by KF and TGA.

Crystalline Form LT1 of Lotilaner may be characterized by each of the above characteristics alone/or by all possible combinations, e.g., an XRPD pattern having peaks at 13.6, 17.4 and 21.4 degrees 2-theta±0.2 degrees 2-theta; an XRPD pattern having peaks at 8.9, 15.0, 21.4, 23.3 and 25.6 degrees 2-theta±0.2 degrees 2-theta an XRPD pattern as depicted in FIG. 2, a solid-state ¹³C NMR spectrum substantially as depicted in FIG. 14, and combinations thereof.

Crystalline Form LT1 as described in any embodiment of the present disclosure may be polymorphically pure, as defined herein above. Moreover, the crystalline Form LT1 as described in any embodiment of the present disclosure may be chemically pure or enantiomerically pure, as defined herein above.

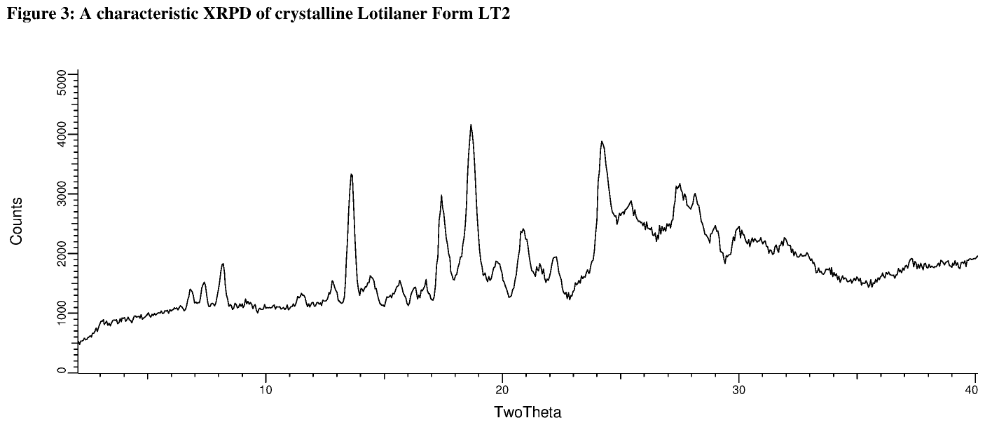

The present disclosure includes a crystalline polymorph of Lotilaner designated Form LT2. The crystalline Form LT2 of Lotilaner may be characterized by data selected from one or more of the following: an X-ray powder diffraction pattern substantially as depicted in FIG. 3; an X-ray powder diffraction pattern having peaks at 13.7, 15.7 and 21.0 degrees 2-theta±0.2 degrees 2-theta; and combinations of these data.

Crystalline Form LT2 of Lotilaner may be further characterized by an X-ray powder diffraction pattern having peaks at 13.7, 15.7 and 21.0 degrees 2-theta±0.2 degrees 2-theta, and also having any one, two, three, four or five additional peaks selected from 7.4, 8.2, 16.4, 19.8 and 24.2 degrees 2-theta±0.2 degrees 2-theta. Crystalline Form LT2 of Lotilaner may be further characterized by an X-ray powder diffraction pattern having peaks at 7.4, 8.2, 13.7, 15.7, 16.4, 19.8, 21.0, and 24.2 degrees 2-theta±0.2 degrees 2-theta.

Alternatively, Crystalline Form LT2 of Lotilaner may be characterized by an X-ray powder diffraction pattern having peaks at 7.4, 8.2, 16.4, 19.8 and 24.2 degrees 2-theta±0.2 degrees 2-theta. Crystalline Form LT2 of Lotilaner may be further characterized by an X-ray powder diffraction pattern having peaks 7.4, 8.2, 16.4, 19.8 and 24.2 degrees 2-theta±0.2 degrees 2-theta and also having any one, two, three, or four additional peaks selected from 13.6, 17.4, 22.3 and 29.0 degrees 2-theta±0.2 degrees 2-theta.

Crystalline Form LT2 of Lotilaner may be characterized by an X-ray powder diffraction pattern having peaks at 7.4, 8.2, 13.6, 16.4, 17.4, 19.8, 22.3, 24.2 and 29.0 degrees 2-theta±0.2 degrees 2-theta.

In one embodiment of the present disclosure, crystalline Form LT2 of Lotilaner is isolated.

Crystalline Form LT2 of Lotilaner may be ethanol solvate. Typically, the ethanol content in LT2 is: about 5.5% w/w to about 7.8% w/w, about 6.0% w/w to about 7.5% w/w, about 6.5% w/w to about 7.2% w/w, or about 6.8% (w/w), as detected for example by KF and TGA.

Crystalline Form LT2 of Lotilaner may be characterized by each of the above characteristics alone/or by all possible combinations, e.g., an XRPD pattern having peaks at 13.7, 15.7, 21.0 degrees 2-theta±0.2 degrees 2-theta; an XRPD pattern having peaks at 7.4, 8.2, 16.4, 19.8 and 24.2 degrees 2-theta±0.2 degrees 2-theta an XRPD pattern as depicted in FIG. 3, and combinations thereof.

Crystalline Form LT2 as described in any embodiment of the present disclosure may be polymorphically pure, as defined herein above. Moreover, the crystalline Form LT2 as described in any embodiment of the present disclosure may be chemically pure or enantiomerically pure, as defined herein above.

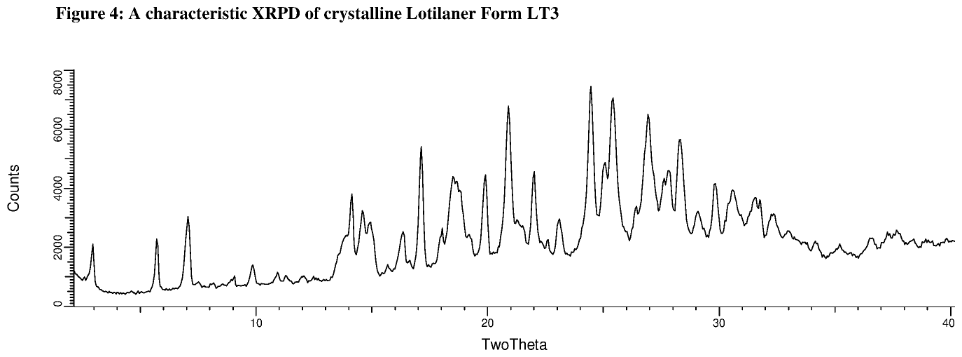

The present disclosure includes a crystalline polymorph of Lotilaner designated Form LT3. The crystalline Form LT3 of Lotilaner may be characterized by data selected from one or more of the following: an X-ray powder diffraction pattern substantially as depicted in FIG. 4; an X-ray powder diffraction pattern having peaks at 13.7, 15.7, 21.0 degrees 2-theta±0.2 degrees 2-theta; and combinations of these data.

Crystalline Form LT3 of Lotilaner may be further characterized by an X-ray powder diffraction pattern having peaks at 13.7, 15.7, 21.0 degrees 2-theta±0.2 degrees 2-theta, and also having any one, two, three, four or five additional peaks selected from 7.1, 13.9, 17.1, 20.9 and 32.4 degrees 2-theta±0.2 degrees 2-theta. Crystalline Form LT3 of Lotilaner may be further characterized by an X-ray powder diffraction pattern having peaks at 7.1, 13.7, 13.9, 15.7, 17.1, 20.9, 21.0, and 32.4 degrees 2-theta±0.2 degrees 2-theta.

Alternatively, Crystalline Form LT3 of Lotilaner may be characterized by an X-ray powder diffraction pattern having peaks at 7.1, 13.9, 17.1, 20.9 and 32.4 degrees 2-theta±0.2 degrees 2-theta. Crystalline Form LT3 of Lotilaner may be further characterized by an X-ray powder diffraction pattern having peaks 7.1, 13.9, 17.1, 20.9 and 32.4 degrees 2-theta±0.2 degrees 2-theta and also having any one, two, three, four or five additional peaks selected from and 2.9, 5.7, 22.0, 24.5 and 29.8 degrees 2-theta±0.2 degrees 2-theta.

Crystalline Form LT3 of Lotilaner may be characterized by an X-ray powder diffraction pattern having peaks at 2.9, 5.7, 7.1, 13.9, 17.1, 20.9, 22.0, 24.5, 29.8 and 32.4 degrees 2-theta±0.2 degrees 2-theta.

In one embodiment of the present disclosure, crystalline Form LT3 of Lotilaner is isolated.

Crystalline Form LT3 of Lotilaner may be a solvate form, for example ethyl acetate solvate. Alternatively, and depending the solvent employed in the procedure, crystalline Form LT3 of Lotilaner may be 1-butanol, 2-methoxyethanol, diglyme, THF, dioxane or acetone solvate. Typically, the solvent content in Form LT3 is: about 4.5% w/w to about 7.2% w/w, about 5% w/w to about 7.0% w/w, or about 7.2% (w/w), as detected for example by KF and TGA. For example, the ethyl acetate content in Crystalline Form LT3 ethyl acetate solvate is of about 7.2% (w/w), as detected by TGA.

Crystalline Form LT3 of Lotilaner may be characterized by each of the above characteristics alone/or by all possible combinations, e.g., an XRPD pattern having peaks at 7.1, 13.9, 17.1, 20.9 and 32.4 degrees 2-theta±0.2 degrees 2-theta; an XRPD pattern having peaks at 13.7, 15.7, 21.0 degrees 2-theta±0.2 degrees 2-theta an XRPD pattern as depicted in FIG. 4, and combinations thereof.

Crystalline Form LT3 as described in any embodiment of the present disclosure may be polymorphically pure, as defined herein above. Moreover, the crystalline Form LT3 as described in any embodiment of the present disclosure may be chemically pure or enantiomerically pure, as defined herein above.

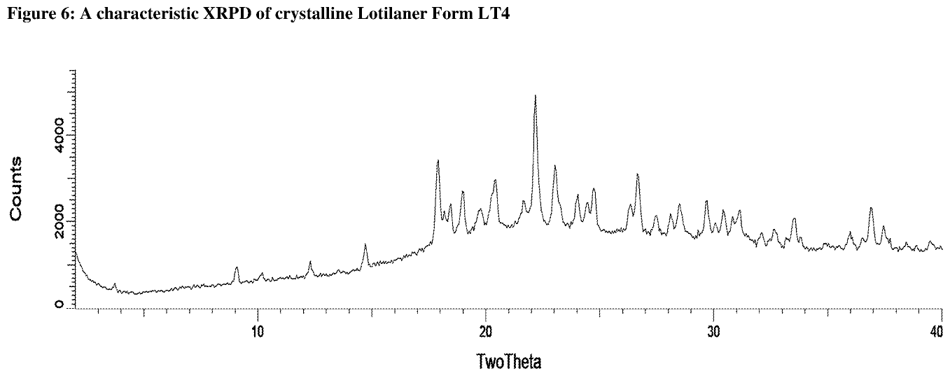

The present disclosure includes a crystalline polymorph of Lotilaner designated Form LT4. The crystalline Form LT4 of Lotilaner may be characterized by data selected from one or more of the following: an X-ray powder diffraction pattern substantially as depicted in FIG. 6; an X-ray powder diffraction pattern having peaks at 12.4, 14.7, 19.0, 22.2 and 26.7 degrees 2-theta±0.2 degrees 2-theta; and combinations of these data.

Crystalline Form LT4 of Lotilaner may be further characterized by an X-ray powder diffraction pattern having peaks at 12.4, 14.7, 19.0, 22.2 and 26.7 degrees 2-theta±0.2 degrees 2-theta, and also having any one, two, three, four or five additional peaks selected from 17.9, 20.4, 23.0 and 29.7 degrees 2-theta±0.2 degrees 2-theta.

Crystalline Form LT4 of Lotilaner may be characterized by an X-ray powder diffraction pattern having peaks at 12.4, 14.7, 17.9, 19.0, 20.4, 22.2, 23.0, 26.7 and 29.7 degrees 2-theta±0.2 degrees 2-theta.

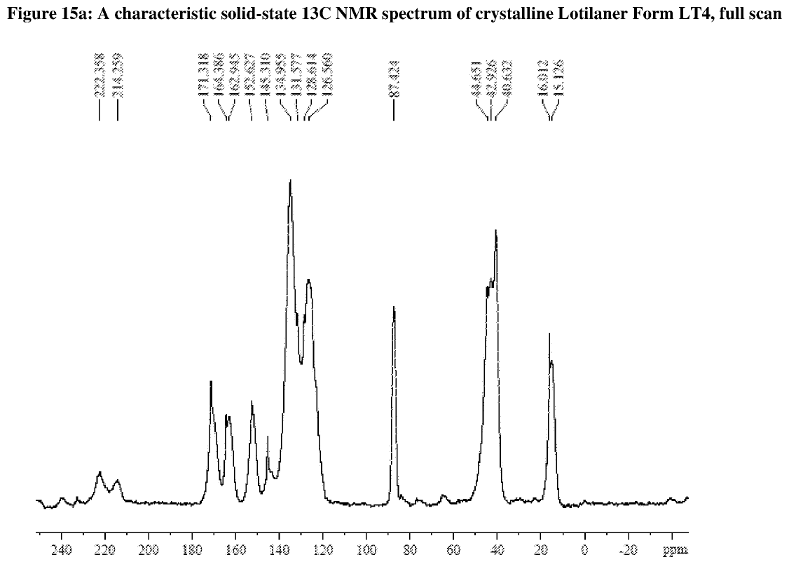

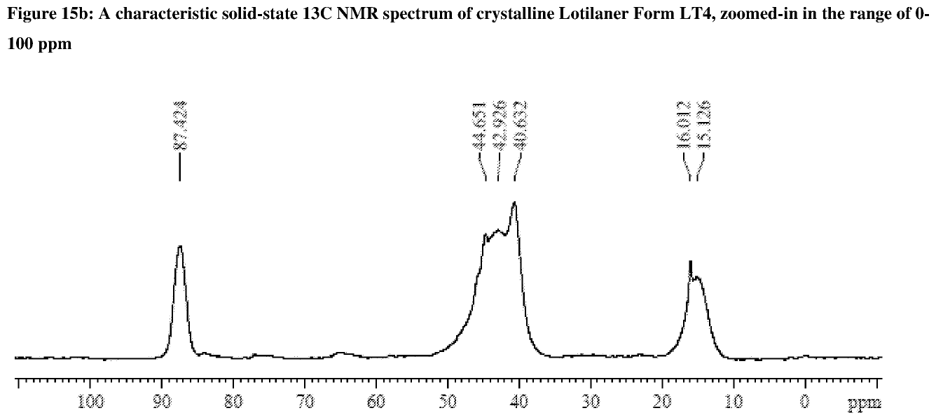

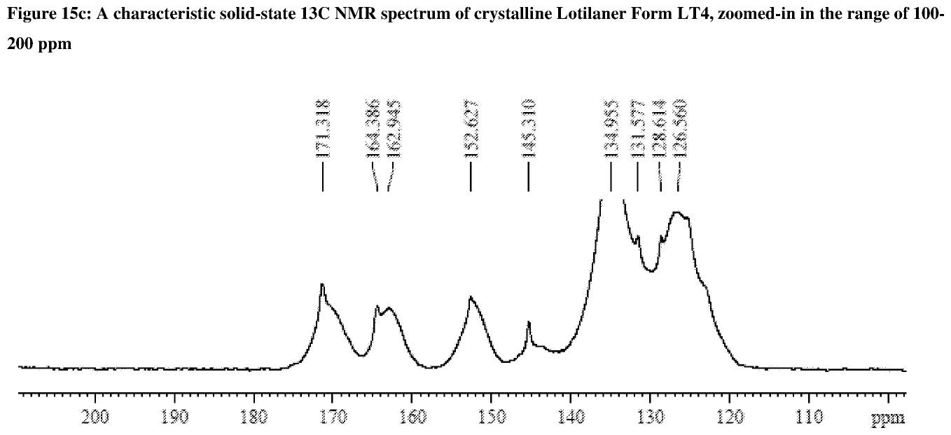

Crystalline Form LT4 of Lotilaner may alternatively or additionally be characterized by a solid state ¹³C spectrum having characteristic peaks at the range of 0-200 ppm at: 16.0, 42.9, 126.6, 135.0, 152.6 and 163.0±0.2 ppm, or a solid-state ¹³C NMR spectrum substantially as depicted in FIG. 15a, 15b or 15c. It may be further characterized by a solid state ¹³C spectrum having characteristic chemical shift absolute differences from a peak at 87.4 ppm±2 ppm of 71.4, 44.5, 39.2, 47.6, 65.2 and 75.6±0.1 ppm; and or by a solid state ¹³C spectrum having characteristic chemical shift differences from a peak at 163.0 ppm±1 ppm of 75.6 ppm±0.1 ppm

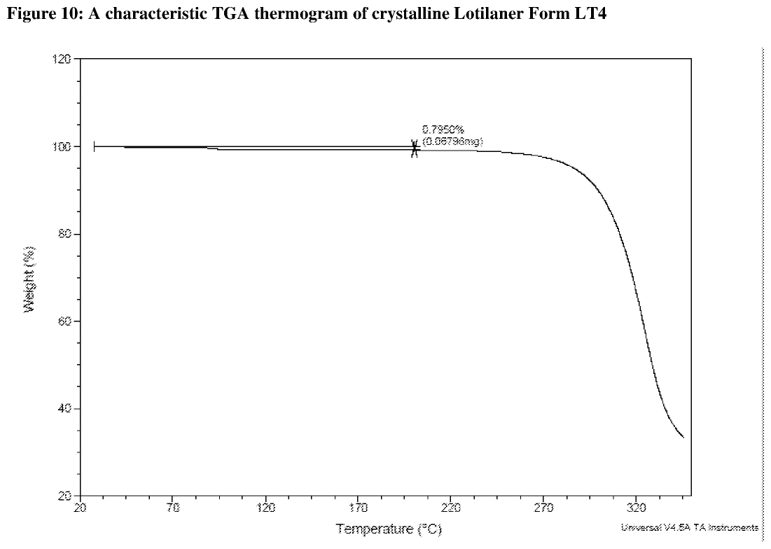

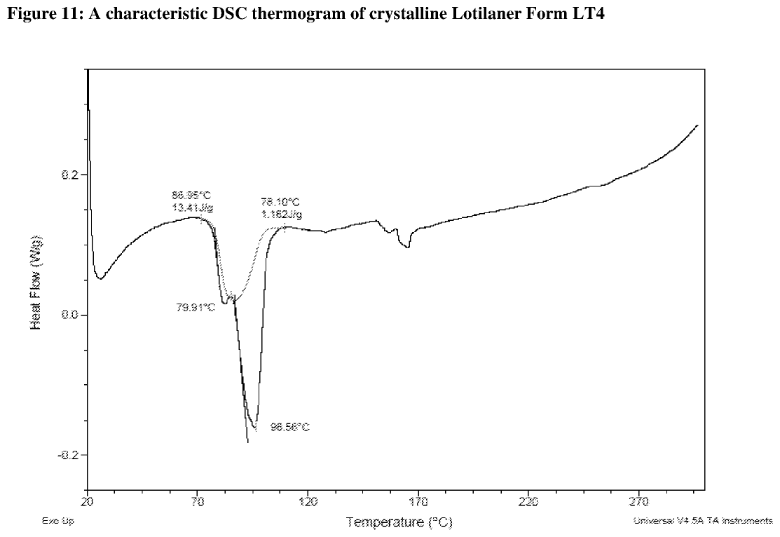

Crystalline Form LT4 of Lotilaner may be characterized by a TGA thermogram substantially as depicted in FIG. 10, or by a DSC thermogram substantially as depicted in FIG. 11, or by combination of these data.

In one embodiment of the present disclosure, crystalline Form LT4 of Lotilaner is isolated.

Crystalline Form LT4 of Lotilaner may be an anhydrous form. Typically, the water content in Form LT4 is less than 1% (w/w), as detected for example by KF and TGA.

Crystalline Form LT4 of Lotilaner may be characterized by each of the above characteristics alone/or by all possible combinations, e.g., an XRPD pattern having peaks at 12.4, 14.7, 19.0, 22.2 and 26.7 degrees 2-theta±0.2 degrees 2-theta; an XRPD pattern as depicted in FIG. 6, and combinations thereof.

Crystalline Form LT4 as described in any embodiment of the present disclosure may be polymorphically pure, as defined herein above. Moreover, the crystalline Form LT4 as described in any embodiment of the present disclosure may be chemically pure or enantiomerically pure, as defined herein above.

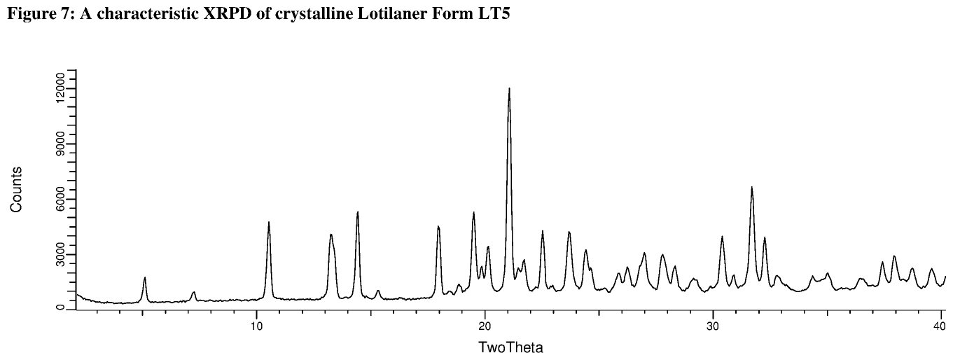

The present disclosure includes a crystalline polymorph of Lotilaner designated Form LT5. The crystalline Form LT5 of Lotilaner may be characterized by data selected from one or more of the following: an X-ray powder diffraction pattern substantially as depicted in FIG. 7; an X-ray powder diffraction pattern having peaks at 5.1, 10.5, 13.3, 21.1 and 23.7 degrees 2-theta±0.2 degrees 2-theta; and combinations of these data.

Crystalline Form LT5 of Lotilaner may be further characterized by an X-ray powder diffraction pattern having peaks at 5.1, 10.5, 13.3, 21.1 and 23.7 degrees 2-theta±0.2 degrees 2-theta, and also having any one, two, three, or four additional peaks selected from 14.4, 18.0, 22.5, and 31.7 degrees 2-theta±0.2 degrees 2-theta.

Crystalline Form LT5 of Lotilaner may be characterized by an X-ray powder diffraction pattern having peaks at 5.1, 10.5, 13.3, 14.4, 18.0, 21.1, 22.5, 23.7 and 31.7 degrees 2-theta±0.2 degrees 2-theta.

Alternatively, Crystalline Form LT5 of Lotilaner may be characterized by an X-ray powder diffraction pattern having peaks at 5.1, 7.2, 10.5, 13.3, 13.9, 14.4, 15.3, 17.4, 18.0, 18.4, 18.9, 19.5, 19.9, 20.2, 21.1, 21.5, 21.7, 22.5, 23.7, 24.4, 24.6, 25.2, 25.9, 26.3, 27.0, 27.8, 28.5, 29.2, 29.8, 30.4, 30.4, 30.9, 31.7, 32.3, 32.8, 34.4, 35.0, 36.5, 37.4, 38.0, 38.7 and 39.6 degrees 2-theta±0.2 degrees 2-theta.

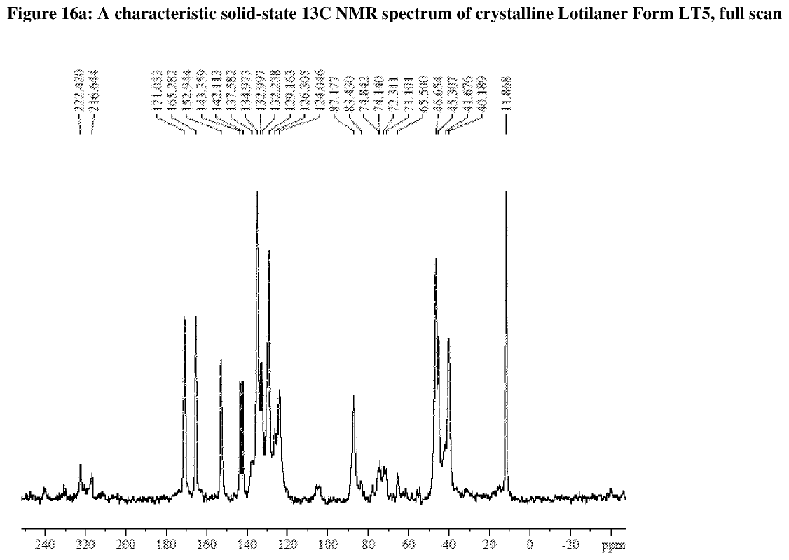

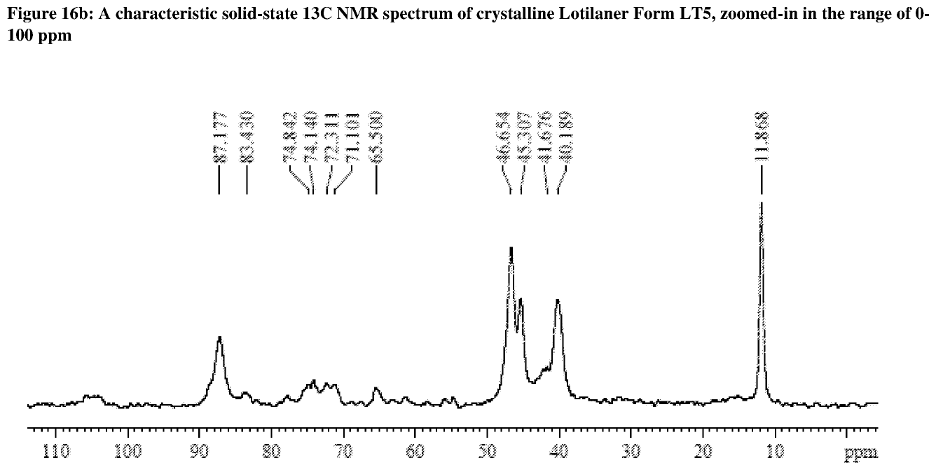

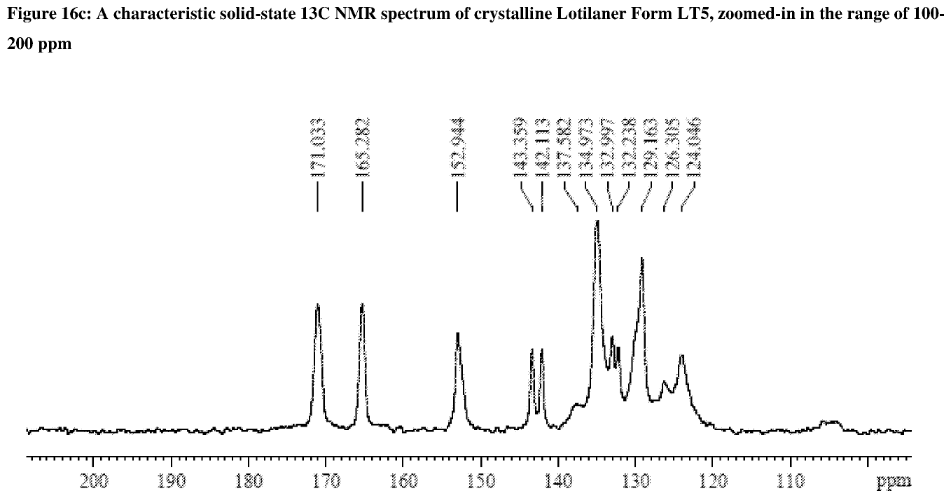

Crystalline Form LT5 of Lotilaner may alternatively or additionally be characterized by a solid state ¹³C NMR spectrum having characteristic peaks at the range of 0-200 ppm at: 11.9, 46.7, 124.1, 129.2, 142.1 and 165.3 ppm±0.2 ppm, or a solid-state ¹³C NMR spectrum substantially as depicted in FIG. 16a, 16b or 16c. It may be further characterized by a solid state ¹³C spectrum having characteristic chemical shift absolute differences from a peak at 87.2 ppm±2 ppm of 75.3, 40.5, 36.9, 42.0, 54.9 and 78.1±0.1 ppm; and or by a solid state ¹³C NMR spectrum having characteristic chemical shift differences from a peak at 165.3 ppm±1 ppm of 78.1±0.1 ppm.

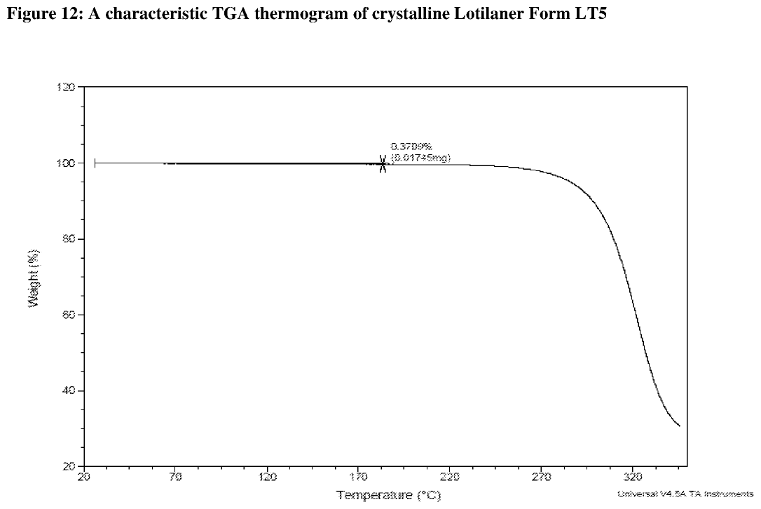

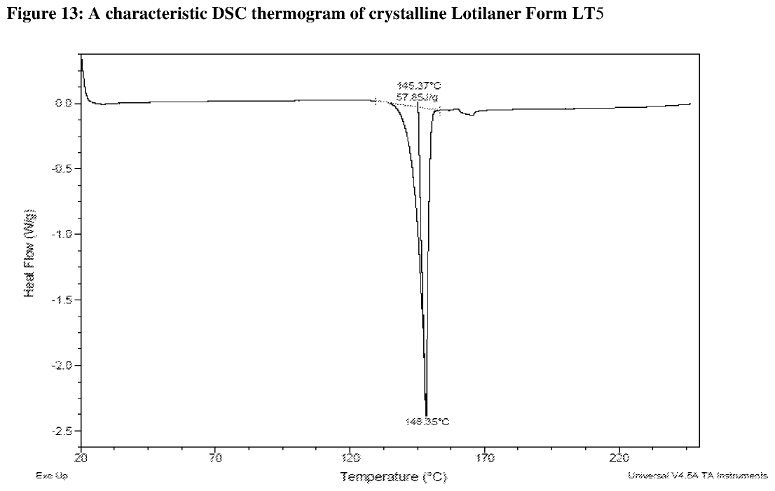

Crystalline Form LT5 of Lotilaner may be characterized by a TGA thermogram substantially as depicted in FIG. 12, a DSC thermogram showing a melting endotherm onset at about 145.4° C.; or by a DSC thermogram substantially as depicted in FIG. 13, or by combination of these data.

In one embodiment of the present disclosure, crystalline Form LT5 of Lotilaner is isolated.

Crystalline Form LT5 of Lotilaner may be an anhydrous form. Typically, the water content in LT5 is less than 1% (w/w), as detected for example by KF and TGA.

Crystalline Form LT5 of Lotilaner may be characterized by each of the above characteristics alone/or by all possible combinations, e.g., an XRPD pattern having peaks at 5.1, 10.5, 13.3, 21.1 and 23.7 degrees 2-theta±0.2 degrees 2-theta; an XRPD pattern as depicted in FIG. 7, and combinations thereof.

Crystalline Form LT5 as described in any embodiment of the present disclosure may be polymorphically pure, as defined herein above. Moreover, the crystalline Form LT5 as described in any embodiment of the present disclosure may be chemically pure or enantiomerically pure, as defined herein above.

The present disclosure further includes processes for preparation of crystalline Form LT5 of Lotilaner, preferably as a polymorphically pure form.

The process may comprise slurrying Lotilaner, preferably form LT3, in a mixture of isopropanol and n-heptane. Typically, the ratio of isopropanol and heptane may be: about 3:1 to about 1:3, about 2:1 to about 1:2, about 1.5:1 to about 1:1.5, about 1.2:1 to about 1:1.2, about 1.1:1 to about 1:1.1, or about 1:1. The process may comprise combining Lotilaner, preferably crystalline form to a mixture of isopropanol and heptane to obtain a slurry, preferably at room temperature. The slurry may be heated, preferably to a temperature of about 40° C. to about 80° C., about 50° C. to about 70° C., about 55° C. to about 65° C., or about 60° C. The slurry may be maintained at the heated temperature for a sufficient time to produce Form LT5. For example, the slurry may be maintained for a period of: about 3 days to about 21 days, about 7 to about 18 days, about 10 to about 16 days, about 12 to about 15 days, or about 14 days. The obtained solid is then isolated, for example by filtration. The isolated solid may be dried, preferably under vacuum, and preferably at a temperature of about 25° C.-30° C., for a sufficient time, for example: about 15 minutes to about 4 hours, about 30 minutes to about 3 hours, about 45 minutes to about 2 hours, or about 1 hour.

Alternatively, the present disclosure provides a process for preparing crystalline Lotilaner form LT5 comprising crystallization of Lotilaner from isoamyl alcohol. The process preferably comprises dissolving Lotilaner in isoamyl alcohol, optionally seeding with Form LT5 of Lotilaner, and cooling. Preferably, the Lotilaner is dissolved in isoamyl alcohol at a temperature of: about 30° C. to about 100° C., about 45° C. to about 80° C., about 50° C. to about 70° C., about 55° C. to about 65° C., or about 60° C. Seeds of crystalline form LT5 are added, to promote the crystallization. Preferably seeds of form LT5 are added. The solution may be cooled down, preferably to a temperature of: about 30° C. to about 50° C., about 35° C. to about 45° C., or about 40° C. prior to adding the seed crystals. The mixture may then be stirred at this temperature for a sufficient time to allow crystals of Form LT5 to form, preferably: about 15 minutes to about 4 hours, about 30 minutes to about 3 hours, about 45 minutes to about 2 hours, or about 1 hour. The mixture may be further cooled to a temperature of: about 20° C. to about 40° C., about 25° C. to about 35° C., or about 30° C. Optionally, the mixture may be stirred at this temperature for: about 15 minutes to about 4 hours, about 30 minutes to about 3 hours, about 45 minutes to about 2 hours, or about 1 hour. The obtained solid may be isolated, for example by filtration. The isolated solid may be dried, preferably under vacuum, at a temperature of about 25° C.-30° C., for a sufficient time, for example about 1 hour.

The solid state forms of Lotilaner as described in any aspect or embodiment of the present disclosure may be polymorphically pure, or substantially free of any other solid state (or polymorphic) forms.

The solid state forms of Lotilaner as described in any aspect or embodiment of the present disclosure may be chemically pure, or substantially free of any other compounds. Particularly, the solid state forms of Lotilaner as described are enantiomerically pure, i.e. substantially free of other enantiomer of Lotilaner.

The present disclosure includes Lotilaner besylate salt, particularly Lotilaner mono-besylate salt. The present disclosure includes crystalline Lotilaner besylate salt, particularly crystalline Lotilaner mono-besylate salt.

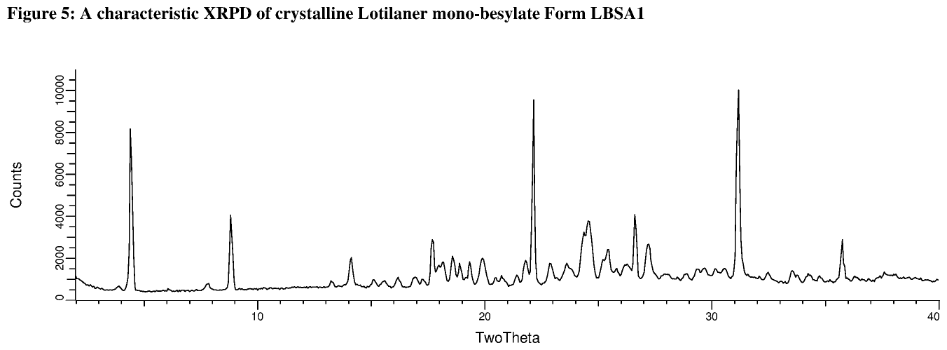

The present disclosure includes a crystalline polymorph Lotilaner mono besylate designated Form LBSA1. The crystalline Form LBSA1 of Lotilaner mono besylate may be characterized by data selected from one or more of the following: an X-ray powder diffraction pattern substantially as depicted in FIG. 5; an X-ray powder diffraction pattern having peaks at 4.4, 8.8 and 14.1 degrees 2-theta±0.2 degrees 2-theta; and combinations of these data.

Crystalline Form LBSA1 of Lotilaner mono besylate may be further characterized by an X-ray powder diffraction pattern having peaks at 4.4, 8.8 and 14.1 degrees 2-theta±0.2 degrees 2-theta, and also having any one, two, three, four or five additional peaks selected from 4.4, 8.8, 14.1, 22.2 and 31.2 degrees 2-theta±0.2 degrees 2-theta. Alternatively, crystalline Form LBSA1 of Lotilaner mono besylate may be characterized by an X-ray powder diffraction pattern having peaks at 4.4, 8.8 and 14.1 degrees 2-theta±0.2 degrees 2-theta, and also having one or both of the additional peaks selected 22.2 and 31.2 degrees 2-theta±0.2 degrees 2-theta.

Alternatively, Crystalline Form LBSA1 of Lotilaner mono-besylate may be characterized by an X-ray powder diffraction pattern having peaks at 4.4, 8.8, 14.1, 22.2 and 31.2 degrees 2-theta±0.2 degrees 2-theta. Crystalline Form LBSA1 of Lotilaner mono-besylate may be further characterized by an X-ray powder diffraction pattern having peaks 4.4, 8.8, 14.1, 22.2 and 31.2 degrees 2-theta±0.2 degrees 2-theta and also having any one, two, three, four or five additional peaks selected from 16.2, 17.7, 19.9, 24.6 and 27.2 degrees 2-theta±0.2 degrees 2-theta.

Crystalline Form LBSA1 of Lotilaner mono-besylate may be characterized by an X-ray powder diffraction pattern having peaks at 4.4, 8.8, 14.1, 16.2, 17.7, 19.9, 22.2, 24.6, 27.2 and 31.2 degrees 2-theta±0.2 degrees 2-theta.

In one embodiment of the present disclosure, crystalline Form LBSA1 of Lotilaner mono-besylate is isolated.

Crystalline Form LBSA1 of Lotilaner mono-besylate may be polymorphically pure.

Further, Crystalline Form LBSA1 of Lotilaner mono-besylate may be chemically pure, and/or enantiomerically pure; preferably it is enantiomerically pure.

Crystalline Form LBSA1 of Lotilaner mono-besylate may be an anhydrous form. Typically, the water content in LBSA1 is less than 1% (w/w), as detected for example by KF and TGA.

Crystalline Form LBSA1 of Lotilaner mono-besylate may be characterized by each of the above characteristics alone or by all possible combinations, e.g., an XRPD pattern having peaks at 4.4, 8.8 and 14.1 degrees 2-theta±0.2 degrees 2-theta; an XRPD pattern having peaks at 4.4, 8.8, 14.1, 22.2 and 31.2 degrees 2-theta±0.2 degrees 2-theta; an XRPD pattern as depicted in FIG. 5, and combinations thereof.

The above solid state forms can be used to prepare other crystalline polymorphs of Lotilaner, Lotilaner salts or co-crystals and their solid state forms.

The present disclosure encompasses a process for preparing other solid state forms of Lotilaner, Lotilaner salts or co-crystals and their solid state forms thereof. The process includes preparing any one of the solid state forms of Lotilaner or of or Lotilaner salts by the processes of the present disclosure, and converting that form to a different form of Lotilaner, Lotilaner salt or co-crystal and solid state forms thereof.

The present disclosure provides the above described solid state forms, particularly crystalline polymorphs of Lotilaner or Lotilaner salts for use in the preparation of pharmaceutical compositions comprising Lotilaner and/or crystalline polymorphs thereof. In embodiments, the above described solid state forms, particularly crystalline polymorphs of Lotilaner or Lotilaner salts are used to prepare a solution or a suspension for ophthalmic administration.

The present disclosure also encompasses the use of the solid state forms, particularly crystalline polymorphs of Lotilaner or Lotilaner salts of the present disclosure for the preparation of pharmaceutical compositions of Lotilaner or Lotilaner salts and/or crystalline polymorphs thereof, particularly ophthalmic solution and/or suspension.

The present disclosure includes processes for preparing the above mentioned pharmaceutical compositions. The processes include combining any one or a combination of the solid state forms, particularly crystalline polymorphs of Lotilaner or Lotilaner salts of the present disclosure with at least one pharmaceutically acceptable excipient.

Pharmaceutical combinations or formulations of the present disclosure contain any one or a combination of the solid state forms of Lotilaner or Lotilaner salts of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g., carbopol), carboxymethylcellulose sodium, dextrin, ethyl cellulose, gelatin, guar gum, hydrogenated vegetable oil, hydroxyethyl cellulose, hydroxypropyl cellulose (e.g., Klucel®), hydroxypropyl methyl cellulose (e.g., Methocel®), liquid glucose, magnesium aluminum silicate, maltodextrin, methylcellulose, polymethacrylates, povidone (e.g., Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Lotilaner and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Lotilaner can be administered. Lotilaner may be formulated for administration to a mammal, in embodiments to a human, by injection or as ophthalmic solution for topical administration. Lotilaner can be formulated, for example, as a viscous liquid solution or suspension, such as a clear solution, for injection or as ophthalmic solution for topical administration. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The solid state forms, particularly crystalline polymorphs of Lotilaner or Lotilaner salts and the pharmaceutical compositions and/or formulations of Lotilaner or Lotilaner salts of the present disclosure can be used as medicaments, in embodiments for the treatment of patients with eye infections and/or blepharitis.

The present disclosure also provides methods of treating of patients with eye infections and/or blepharitis, by administering a therapeutically effective amount of any one or a combination of the solid state forms, particularly crystalline polymorphs of Lotilaner or Lotilaner salts of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-ray Diffraction (“XRPD”) Method

X-ray diffraction was performed on X-Ray powder diffractometer Bruker D8 Advance; CuK_radiation (λ=1.5418 Å); Lynx eye detector; laboratory temperature 22-25° C.; PMMA specimen holder ring. Prior to analysis, the samples were gently ground by means of mortar and pestle in order to obtain a fine powder. The ground sample was adjusted into a cavity of the sample holder and the surface of the sample was smoothed by means of a cover glass.

### Measurement Parameters:

Scan range: 2-40 degrees 2-theta;
Scan mode: continuous;
Step size: 0.05 degrees;
Time per step: 0.5 s;
Sample spin: 30 rpm;
Sample holder: PMMA specimen holder ring with silicon low background holder.

X-Ray Powder Diffraction peak values were calibrated with respect to standard silicon spiking in the sample.

### Solid State ¹³C-NMR Method:

The solid-state NMR spectra are measured at 11.7 T using a Bruker Avance III HD 500 US/WB NMR spectrometer (Karlsruhe, Germany, 2013) with a 4-mm probe head.

The ¹³C CP/MAS NMR spectra employing cross-polarization are acquired using the standard cross-polarization pulse scheme at spinning frequency of 11 kHz. The cross-polarization contact time is usually 2 ms, and the dipolar decoupling SPINAL64 is applied during the data acquisition. The number of scans is set for the signal-to-noise ratio SINO reaches at least the value ca. 50. The 13C scale is referenced to a-glycine (176.03 ppm for 13C).

Frictional heating of the spinning samples is compensated by active cooling, and the temperature calibration is performed with Pb(NO3)₂.

### TGA Method:

Thermogravimetric analysis was conducted on a TA instrument Q500 thermogravimetric analyzer. About 4-8 mg sample was placed into a tared TGA crucible and placed into a TGA furnace. The furnace was heated under nitrogen at a heating rate of 10° C./min up to 350° C.

### DSC Method:

DSC was performed using a TA instrument Q2000 differential scanning calorimetry. About 1.0-3.0 mg sample was accurately weighed into an aluminum pan, covered with a lid and crimped. After crimp the aluminum lid was pin holed using needle. The sample cell was equilibrated at 20° C. and heated at a rate of 10° C./min up to 300° C. under the nitrogen atmosphere.

### EXAMPLES

### Preparation of Starting Materials

Lotilaner can be prepared according to methods known from the literature, for example according to the disclosure in International Publication No. WO 2010/070068, for example according to the procedure described in Example 4.

### Example 1: Preparation of Amorphous Lotilaner

Lotilaner (3.0 grams) was dissolved in dichloromethane (30 mL) at a temperature of about 25° C.-30° C. The solution was filtered through 0.45-micron filter. The clear solution was subjected to distillation under reduced pressure on a rotary evaporator at 35° C.-40° C. for 30-45 minutes. The obtained solid was analyzed by XRPD, amorphous Lotilaner was obtained. The XRPD pattern is presented in FIG. 1.

### Example 2: Preparation of Lotilaner Crystal Form LT1

Lotilaner (Amorphous, 0.25 grams) was dissolved in dichloromethane (1.0 mL) at a temperature of about 25° C.-30° C. The obtained solution was filtered through 0.45 micron and the clear solution was covered with parafilm with a pin hole and was kept for slow solvent evaporation at 25° C.-30° C. for about 24 hours. After one day, the solid was isolated and analyzed by XRPD. Crystalline Lotilaner Form LT1 was obtained, The XRPD pattern is presented in FIG. 2.

### Example 3: Preparation of Lotilaner Crystal Form LT2

Lotilaner (Amorphous, 0.1 grams) was dissolved in ethanol (0.5 ml) at temperature of about 25-30° C. The obtained solution was filtered through a 0.45 micron filter and the obtained clear solution was covered with parafilm with a pin hole and was kept for slow solvent evaporation at 25-30° C. After two days, the solid was isolated and analyzed by XRPD. Crystalline Lotilaner Form LT2 was obtained, The XRPD pattern is presented in FIG. 3.

Form LT2 obtained in this example is an ethanol solvate.

### Example 4: Preparation of Lotilaner Crystal Form LT3

Lotilaner (Amorphous, 0.1 grams) was dissolved in ethyl acetate (0.2 ml) at temperature of about 60° C. The obtained clear solution was cooled down to 0-5° C. under stirring. Then, it was maintained at temperature of about 25° C. for about 2 hours. The obtained solid was isolated and analyzed by XRPD. Crystalline Lotilaner Form LT3 was obtained, The XRPD pattern is presented in FIG. 4.

Form LT3 obtained in this example is an ethyl acetate solvate.

The above described process was performed using different solvent according to the following list: 1-butanol, 2-methoxyethanol, diglyme, THF, dioxane solvate or acetone.

Depending which solvent used, Form LT3 may also be a 1-butanol, 2-methoxyethanol, diglyme [also known as bis(2-methoxyethyl) ether], THF, dioxane solvate or an acetone solvate.

### Example 5: Preparation of Lotilaner Crystal Form LT3

Lotilaner (0.1 grams) was dissolved in Tetrahydrofuran (THF, 0.3 ml) at temperature of about 60° C. The obtained clear solution was maintained at 60° C. and then water (0.9 ml) was added and the mixture was stirred for about 30 minutes. The reaction mixture was then allowed to reach temperature of about 25° C. and it was maintained at this temperature for about 18 hours. The solid was filtered, washed with water (2 ml×3) and dried under vacuum for 15-30 minutes. The obtained solid was analyzed by XRPD, crystalline Lotilaner Form LT3 was obtained, Form LT3 obtained in this example is THF solvate.

The above described process was performed using different solvent according to the following list: Diglyme, ethyl acetate or dioxane solvate. Depending which solvent used, Form LT3 prepared in this process may also be diglyme, ethyl acetate or dioxane solvate.

### Example 6: Preparation of Lotilaner Mono-Besylate Crystal Form LBSA1

Lotilaner (form LT1, 0.25 grams) was dissolved in ethanol (2.0 mL) at temperature of about 25-30° C. A solution of benzene sulfonic acid was prepared by dissolving benzene sulfonic acid (about 0.08 grams) in ethanol (1 ml) at 25° C. The Benzoic acid solution was slowly added into the Lotilaner solution at 25° C. and maintained under stirring for 4 hours. The obtained clear solution was further cooled down to a temperature of about 0° C. Then, heptane (10 ml) was added to the solution and the obtained mixture was maintained at temperature of about 0° C. under stirring for about 7 days. The mixture was then allowed to reach room temperature (25° C.) and the solid was filtered. The obtained solid was washed with heptane and dried under vacuum for 30 minutes. The solid was isolated and analyzed by XRPD. Crystalline Lotilaner mono-besylate Form LBSA1 was obtained, The XRPD pattern is presented in FIG. 5.

### Example 7: Preparation of Lotilaner Crystal Form LT4

Lotilaner (0.45 grams, amorphous) was dissolved in isopropyl alcohol (2 ml) at temperature of about 25° C. The obtained solution was filtered through 0.45 micron filter and the clear stock solution (1.8 ml) was added into precooled water (8 ml, pre-cooled to temperature of about 0-5° C.) under stirring. The reaction mixture was maintained for about 1-2 hours and filtered under vacuum for about 15-20 minutes. The obtained solid was dried under vacuum at a temperature of about 25-30° C. for about 1 hour. The sample was further dried under vacuum oven at a temperature of about 60° C. for 2 hours. The obtained solid was analyzed by XRPD, Crystalline Lotilaner Form LT4 was obtained, The XRPD pattern is presented in FIG. 6.

The above described process was performed using ethanol/water system in the same volume and conditions described above, form LT4 was obtained.

### Example 8: Preparation of Lotilaner Crystal Form LT5

Lotilaner (Form LT3, 0.03 grams) was added into a mixture of isopropanol and heptane (1:1 v/v, total volume 0.5 ml) at a temperature of about 25° C. The slurry was maintained under stirring at a temperature of about 60° C. for 14 days. The reaction mixture was filtered under vacuum for about 5-10 minutes. The obtained solid was dried under vacuum at a temperature of about 25-30° C. for about 1 hour. The solid was isolated and was analyzed by XRPD and designated as Form LT5 of Lotilaner, an anhydrous form.

### Example 9: Preparation of Lotilaner Crystal Form LT5

Lotilaner (0.05 grams) was dissolved in isoamyl alcohol (0.2 ml) at a temperature of about 60° C. The obtained solution was obtained under stirring. Seeds of form LT5 (1 mg, which is about 2% w/w of input sample were added into the solution at a temperature of about 40° C. under stirring and it was further maintained for about 1 hour, Then, the reaction mixture was cooled down to a temperature of about 30° C. (cooling rate of about 1° C. per minute) and maintained for 1 hours under stirring at a temperature of about 30° C. The reaction mixture was filtered under vacuum for about 5-10 minutes. The obtained solid was washed with heptane (1 ml×3) and was dried under vacuum at temperature of about 25-30° C. for about 1 hour. The solid was isolated and analyzed by XRPD and designated as Form LT5 of Lotilaner. An XRPD pattern is presented in FIG. 7. Form LT5 is an anhydrous form.

### Example 10: Stability Experiments

### Storage Stability at Different Relative Humidities

Samples of Lotilaner forms were subjected to conditions of different relative humidities at ambient temperature. XRPD analysis was performed on the samples after 7 days. The results are shown in Table 1 below:

TABLE 1

| | Relative humidity | | | | |
|---|---|---|---|---|---|
| Sample | 20% | 40% | 60% | 80% | 100% |
| Form LT1 Lotilaner | No | No | No | No | No |
| | change | change | change | change | change |
| Form LT4 Lotilaner | No | No | No | No | No |
| | change | change | change | change | change |
| Form LT5 Lotilaner | No | No | No | No | No |
| | change | change | change | change | change |

The results demonstrate that forms LT1, LT4 and LT4 are stable after exposure to high and low relative humidity for at least 7 days.

Samples of Lotilaner forms were subjected to conditions of different temperatures and/or relative humidities. XRPD analysis was performed on the samples after 1, 3 or 6 months. The results are shown in Table 2 below:

TABLE 2

| | Conditions | | |
|---|---|---|---|
| | | 25° C., 60% | 40° C., 75% |
| Sample | 2-8° C. | RH | RH |
| Form LT1 Lotilaner (6 m) | No change | No change | No change |
| Form LT4 Lotilaner (3 m) | No change | No change | No change |
| Form LT5 Lotilaner (1 m) | No change | No change | No change |

The results demonstrate that Forms LT1, LT4 and LT5 have good storage stability.

### Grinding Experiments

Samples of Lotilaner forms were subjected to strong grinding, and to solvent drop grinding in water, ethanol or isopropanol. Grinding was carried out on the samples alone, or in the presence of ethanol, water or isopropanol. In these experiments, about 20 mg of the sample is placed in a mortar and ground with a pestle for 2 minutes. The solvent, when used, as added to the crystalline material before grinding, in a volume of 10 microlitres. XRPD analysis performed on each of the samples after the grinding experiment, confirmed no change in the starting material (Table 3):

TABLE 3

| | Form LT1 | Form LT4 | Form LT5 |
|---|---|---|---|
| Condition | Lotilaner | Lotilaner | Lotilaner |
| Strong grinding | No change | No change | No change |
| Solvent-drop grinding | No change | ND | No change |
| (ethanol) | | | |
| Solvent-drop grinding | No change | No change | No change |
| (water) | | | |
| Solvent-drop grinding | No change | No change | No change |
| (isopropanol) | | | |

The results demonstrate that Lotilaner Forms LT1, LT4 and LT5 are resistant to polymorphic changes and is highly suitable for preparing pharmaceutical formulations.

### Thermal Stability

Samples of Lotilaner forms were subjected to heating up to 100° C. for 30 minutes. XRPD analysis of the sample confirmed no change in the starting material (Table 4):

TABLE 4
Heating 100° C., 30 minutes

| Form LT1 Lotilaner | Form LT4 Lotilaner | Form LT5 Lotilaner |
|---|---|---|
| No change | No change | No change |

### Stability to Compression

Samples of Lotilaner forms were subjected to pressures of 2 tons for one minute. XRPD analysis was performed on the samples after 1 minute. The results are shown in Table 5 below:

TABLE 5

| XRPD analysis results | Pressure (2 tons) |
|---|---|
| Lotilaner Form LT1 | No change |
| Lotilaner Form LT4 | No change |
| Lotilaner Form LT5 | No change |

Accordingly, Lotilaner Forms LT1, LT4 and LT5 are stable under high pressure conditions, making it highly suitable for pharmaceutical processing.

1-53. (canceled)

54. A pharmaceutical composition for ophthalmic administration comprising at least one pharmaceutically acceptable excipient in combination with a Crystalline Lotilaner designated form LT5, which is characterized by data selected from:
a) an X-ray powder diffraction pattern substantially as depicted in FIG. 7;
b) an X-ray powder diffraction pattern having peaks at 5.1, 10.5, 13.3, 21.1 and 23.7 degrees 2-theta±0.2 degrees 2-theta;
c) a solid state ¹³C spectrum having characteristic peaks at the range of 0-200 ppm at: 11.9, 46.7, 124.1, 129.2, 142.1 and 165.3 ppm±0.2 ppm,
d) a solid-state ¹³C NMR spectrum substantially as depicted in FIG. 16a, 16b or 16c;
e) solid state ¹³C spectrum having characteristic chemical shift absolute differences from a peak at 87.2 ppm±2 ppm of 75.3, 40.5, 36.9, 42.0, 54.9 and 78.1±0.1 ppm;
f) a solid state ¹³C NMR spectrum having characteristic chemical shift differences from a peak at 165.3 ppm±1 ppm of 78.1±0.1 ppm; and
g) a combination of two or more or (a), (b), (c), (d), (e) and (f).

55. The pharmaceutical composition of claim 54, wherein the Crystalline Lotilaner designated form LT5 is characterized by an X-ray powder diffraction pattern having peaks at 5.1, 10.5, 13.3, 21.1 and 23.7 degrees 2-theta±0.2 degrees 2-theta, and also having any one, two, three, or four additional peaks selected from 14.4, 18.0, 22.5, and 31.7 degrees 2-theta±0.2 degrees 2-theta.

56. The pharmaceutical composition of claim 54, wherein the Crystalline Lotilaner designated form LT5 is characterized by an X-ray powder diffraction pattern having peaks at 5.1, 10.5, 13.3, 14.4, 18.0, 21.1, 22.5, 23.7 and 31.7 degrees 2-theta±0.2 degrees 2-theta.

57. The pharmaceutical composition of claim 54, wherein the Crystalline Lotilaner designated form LT5 is characterized by an X-ray powder diffraction pattern having peaks at 5.1, 7.2, 10.5, 13.3, 13.9, 14.4, 15.3, 17.4, 18.0, 18.4, 18.9, 19.5, 19.9, 20.2, 21.1, 21.5, 21.7, 22.5, 23.7, 24.4, 24.6, 25.2, 25.9, 26.3, 27.0, 27.8, 28.5, 29.2, 29.8, 30.4, 30.4, 30.9, 31.7, 32.3, 32.8, 34.4, 35.0, 36.5, 37.4, 38.0, 38.7 and 39.6 degrees 2-theta±0.2 degrees 2-theta.

58. The pharmaceutical composition of claim 54, wherein the Crystalline Lotilaner designated form LT5 is characterized by a TGA thermogram substantially as depicted in FIG. 12, a DSC thermogram showing a melting endotherm onset at about 145° C., a DSC thermogram substantially as depicted in FIG. 13, or by combinations of these data.

59. The pharmaceutical composition of claim 54, wherein the pharmaceutical formulation is an ophthalmic solution or ophthalmic suspension.

60. A process for preparing the pharmaceutical composition according to claim 54, comprising combining the Crystalline Lotilaner designated form LT5 with the at least one pharmaceutically acceptable excipient.

61. A method for treating eye infections and/or blepharitis, comprising administering the pharmaceutical composition according to claim 54 to a subject in need of the treatment.

62. A process for preparing a Crystalline Lotilaner designated form LT5, comprising combining a Lotilaner in a mixture of isopropanol and n-heptane to form a slurry.

63. The process according to claim 62, wherein the ratio of isopropanol to n-heptane is from about 3:1 to about 1:3.

64. The process of claim 62, wherein the Lotilaner is form LT3.

65. The process according to claim 62, wherein the Lotilaner is combined with the mixture of isopropanol and heptane at room temperature.

66. The process according to claim 62, wherein the slurry is heated to a temperature from about 40° C. to about 80° C.

67. The process according to claim 66, wherein the slurry is heated for a time from about 3 days to about 21 days.

68. A process for preparing a Crystalline Lotilaner designated form LT5, comprising crystallization of a Lotilaner from isoamyl alcohol.

69. The process according to claim 68, comprising dissolving the Lotilaner in isoamyl alcohol to form a solution, optionally seeding with the Crystalline Lotilaner designated form LT5, and cooling.

70. The process according to claim 69, wherein the Lotilaner is dissolved in the isoamyl alcohol at a temperature from about 30° C. to about 100° C.

71. The process according to claim 69, wherein the solution is cooled to a temperature from about 30° C. to about 50° C.

72. The process according to claim 69, wherein the solution is stirred for a time from about 15 minutes to about 4 hours to allow crystals of the Crystalline Lotilaner designated form LT5 to form.

73. The process according to claim 69, wherein the solution is further cooled to a temperature from about 20° C. to about 40° C.

74. The process according to claim 73, wherein the solution is stirred for a time from about 15 minutes to about 4 hours.

75. The process according to claim 68, wherein the Crystalline Lotilaner designated form LT5 is isolated by filtration.

76. The process according to claim 75, wherein the Crystalline Lotilaner designated form LT5 is dried at a temperature from about 25° C. to about 30° C. for a time from about 15 minutes to about 4 hours.
